(19) United States
(12) Patent Application Publication
(10) Pub. No.: US 2006/0058398 A1
(43) Pub. Date: Mar. 16, 2006

(54) Medicinal compositions containing vitamin k's as nerve growth factor potentiator and utilization thereof

(75) Inventors: Yuto Kamei, Saga (JP); Takeshi Goto, Ibaraki (JP); Hidenao Fukushima, Ibaraki (JP); Kengo Uemura, Ibaraki (JP)

Correspondence Address: WOLF GREENFIELD & SACKS, PC;FEDERAL RESERVE PLAZA, 600 ATLANTIC AVENUE, BOSTON, MA, 02210-2211, US

(73) Assignee: Hisamitsu Pharmaceutical Co., Inc., Tosy-shi Saga (JP)

(21) Appl. No.: 10/502,836

(22) PCT Filed: Jan. 24, 2003
(86) PCT No.: PCT/JP03/00650

§ 371 Date: Mar. 28, 2005

(30) Foreign Application Priority Data
Jan. 31, 2002 (JP) 2002-22815

(51) Int. Cl. A61K 31/12

(52) U.S. Cl. 514/682

(57) ABSTRACT

To provide medicinal compositions containing an active ingredient which has a low molecular weight, is free from any side effects and has an effect of potentiating nerve growth factor activity and utilization thereof for treating diseases associated with dementia, medicinal preparations containing an effective amount of vitamin K and/or its derivative showing an effect of potentiating nerve growth factor activity are provided.

### TECHNICAL FIELD

The invention relates to a medicinal composition containing vitamin K and its derivative showing an effect of potentiating nerve growth factor and utilization thereof for diseases associated with dementia.

### BACKGROUND ART

The hypofunction in the brain is recognized from two points of views, which are reduction in the number of nerve cells and disappearance of their network. The network of nerve cells is formed in such a way that dendrites elongating from a nerve cell combine with dendrites, axial filaments or cell bodies in many nerve cells and constitute synapses, making various information treatments including memory, learning and perception possible. Although usually several ten thousand synapses per one nerve cell exist, a remarkable reduction of dendrites occurs in a nerve cell which is aged or damaged. As the result, the number of synapses decreases and thus lowering of a brain function is caused. Then, cells aged or damaged, in which the atrophy and degeneration of dendrites or axial filaments steadily progress, finally die.

Although reduction of nerve cells and change of a synapse network steadily progress, accompanied by aging or the progress of disease, this change does not always cause lowering of the function. For example, it is known that even if a part of the nerve cell network is broken, nerve cells remaining in the neighborhood elongate neurites whereby a new synapse is formed, having a function to compensate to some degree the lowered function of the nerve circuit network by nerve drop out.

Currently, the tendency of elderly population increase is becoming more serious in developed countries, and it is anticipated that also in our country the proportion of elderly aged people in the total population will become the top in the world. Because of the increase of senile dementia accompanied by aging as such, especially of Alzheimer type dementia being an intractable disease, which is progressively lethal, with a pathological characteristics such as degeneration-dropout or senile plaque, research on Alzheimer type dementia has been carried out extensively to date, making a lot of facts become clear. For example, facts such as the appearance of abnormal structural bodies including senile plaque (Masters et al., Proc. Natl. Acad. Sci. USA, 82, 4245-4249, 1985; Seuber et al., Nature, 359, 325-327, 1992), Alzheimer's fibrillary (Kondo et al., Neuron, 1, 827-834, 1988) or the like, accumulation of aluminum in the brain (Kawahara et al., Koushu Eisei Kenkyu, 42, 520-525, 1993) and deficit of neurotrophic factor (Hefti et al., Ann. Neurol., 13, 109-110, 1983) have been made clear. However, even now a clear elucidation of the pathogenesis or a therapeutic method have not been established yet, so that the elucidation of the pathogenesis and establishment of the therapeutic method is an urgent problem.

Further, in addition to the above described Alzheimer senile dementia, illustrative of disease accompanying dementia are such as juvenile Alzheimer dementia, Pick disease, Huntington chorea, Parkinson disease, spinocerebelar degeneration, progressive supranuclear palsy and intractable epilepsy, which are classified as nerve degenerative diseases, multiple cerebral infarction, Binswanger type subcortical encephalopathy and chronic subdural hematoma, which are classified as angiopathic diseases, multiple sclerosis and collagen disease, which are classified as infectious diseases, primary brain tumor, metastatic brain tumor and limbic encephalitis, which are classified as brain tumors, and manic-depressive psychosis, schizoid and reactive psychosis, which are classified as mental diseases.

The neurotrophic factor is a physiologically active substance with a diffusing property, necessary for differentiation and survival sustainment for nerve cells (Thoenen & Edgar, Science, 229, 238-242, 1985), and almost all of them are glycoprotein. It is considered that each neurotrophic factor is synthesized in a projecting site of each nerve cell's axial filament, that is, glia cell or a vicinal cell, and is taken in the cell body from the end of the axial filament by antiport, showing various pharmacological actions.

The first neurotrophic factor found is Nerve Growth Factor (NGF, Thoenen & Brade, Physiol. Rev., 60, 1284-1335, 1980) acting to sympathetic nerves, sensory nerves at a fetal early period, or cholinergic nerve cells in the frontal basal nuclei. After the finding of NGF reseach of a neurotrophic factor was extensively carried out, and Brain Derived Neurotrophic Factor acting to sensory nerve cells insensitive toward NGF (BDNF; Brade et al., EMBO. J. 1, 549-553, 1982), Cilialy Neurotrophic Factor acting to parasympathetic nerves (CNTF; Watters & Hendy, J. Neurochem., 49, 705-713, 1987), further, Neurotrophin-3 belonging to these gene families (NT-3; Hohn et al., Nature, 344, 339-341, 1990; Maisonpierre et al., Science, 247, 1446-1451, 1990), Neurotrophin-4 (NF-4) and Neurotrophin-5 (NF-5) (Davies et al., J. Neurosci., 13, 4961-4967, 1993), etc., were newly found. Further, it has been made evident that Fibroblast Growth Factor (FGF; Morrison et al., Proc. Natl. Acad. Sci. USA, 83, 7537-7541, 1986), Epidermal Growth Factor (EGF; Morrison et al., Science, 238, 72-75, 1987) and Insulin-Like Growth Factor (Aizerman & Vellis, Brain. Res. 406, 32-42, 1987), which are the known cell growth factors, also keep the survival of nerve cells under incubation, making a neurotrophic factor like action. Additionally, besides these glycoproteins it is reported that thyroxine of thyroid hormone which is the amino acid containing iodine (Hayashi & Payel, Dev. Brain. Res., 36, 109-120, 1987) and estradiol which is a steroid hormone (Arimatsu & Hatanaka, Dev. Brain. Res., 26, 151-159, 1986) also show a neurotrophic factor like action. Thus, it is an unquestionable fact that various neurotrophic factors are necessary for keeping the function or survival of nerve cells.

According to the neurotrophic factor deficiency theory of Hefti et al., (Ann. Neurol., 36, 109-120, 1987) it is considered that the degeneration-atrophy of nerve cells due to reduction of neurotrophic factor synthesis ability involved in the survival and differentiation acceleration of nerve cells, or the dropout of nerve cells due to the apoptosis or necrosis of nerve cells occurs, causing the onset of Alzheimer type dementia. In these circumstances, it was proved that NGF, one of neurotrophic factors, acts to large cholinergic nerve cells of cerebral Meynert nuclei, showing survival and differentiation acceleration action of nerve cells (Whitehouse et al., Science, 215, 1237-1239, 1982; Bartus et al., Science, 217, 408-417, 1982; Fibiger et al., TINS, 14, 220-223, 1991), and it was suggested that there is a close relationship between NGF action and Alzheimer type dementia (Brade, Neuron, 2, 1525-1534, 1989). Actually, when NGF is administered directly into the brain of Alzheimer type dementia patient, the disease condition is recovered transiently (Olson et al., J. Neural. Transm., 4, 79-95, 1992).

However, because NGF is a glycoprotein having molecular weight of 140,000, it can not pass through blood-brain barrier, and there are problems such as its absorption efficiency or stability in the blood or digestive tract. Therefore, it is anticipated that use of NGF itself as a pharmaceutical preparation is extremely difficult.

Currently, development of drugs to treat the disease has been tried, and among them an acetyl choline esterase inhibitor is being used as an anti-dementia agent, because reduction of acetyl choline content in cerebral cortex projection nerve system making Meynert nuclei as nucleus originis is observed at the onset. However, although it is possible to some degree to delay the progress of Alzheimer type dementia, due to the fact that reduction of brain function accompanied with nerve cell death is irreversible, it is extremely difficult to improve the disease conditions and it is whatever only a symptomatic treatment. Further, since acetyl choline is a neurotransmitter in the parasympathetic nerve system, not only side effects related to circulatory organs such as syncope, bradycardia, cardiac block or myocardial infarction but severe side effects such as peptic ulcer, hepatopathy, hepatitis or cerebral attack are concerned.

Under this background, it has been desired to provide a low molecular compound, which increases NGF activity, has a molecular size possible for passing through the blood-brain barrier and is reduced in side effects.

Consequently, the object of the invention is to provide a medicinal composition containing an active ingredient which has a low molecular weight, is free from any side effects and has an effect of potentiating nerve growth factor activity and utilization thereof for treating disease associated with dementia.

Meanwhile, the medicinal composition having an effect of potentiating nerve growth factor activity in the invention includes a composition having a neurite elongation action, brain function activating action, memory improvement or anti-dementia actions, or the like in a broader sense, and preparations containing said composition obviously mean a neurite elongation, brain function activation, memory improvement or anti-dementia agents.

### DISCLOSURE OF THE INVENTION

The inventors searched various kinds of artificial synthetic compounds and natural products as low molecular weight compounds with low side effects as described above, and as the results found out that as the latter examples, that is, natural products, dodecatrienoic acid derivatives have an effect of potentiating nerve growth factor activity (WO 01/58437).

Under the above information, the inventors made an extensive research in view of the above problems and as the results found out that vitamin K, which was usually known having a blood-coagulation effect, but which had not been known to have an effect for the nervous system, has an effect of potentiating nerve growth factor activity and treatment of diseases associated with dementia becomes possible by a composition having an appropriate dosage form containing it as an active ingredient, and accomplished the invention.

Namely, the invention relates to a medicinal composition containing an effective amount of vitamin K and/or its derivative showing an effect of potentiating nerve growth factor activity.

Also, the invention relates to the above medicinal composition wherein vitamin K and/or its derivative (vitamin K's) is one or more selected from vitamin K1, vitamin K2 or derivatives thereof.

Further, the invention relates to the above medicinal composition wherein the amount of vitamin K and/or its derivative showing an effect of potentiating nerve growth factor activity is 0.005-50 wt. %.

Furthermore, the invention relates to utilization of the above medicinal composition for treatment of diseases associated with dementia.

Additionally, the invention relates to utilization of the above medicinal composition wherein the diseases associated with dementia are selected from a group consisting of Alzheimer senile dementia, juvenile Alzheimer dementia, Pick disease, Huntington chorea, Parkinson disease, spinocerebelar degeneration, progressive supranuclear palsy, intractable epilepsy, multiple cerebral infarction, Binswanger type subcortical encephalopathy, chronic subdural hematoma, multiple sclerosis, collagen disease, primary brain tumor, metastatic brain tumor, limbic encephalitis, manic-depressive psychosis, schizoid and reactive psychosis.

Since vitamin K's used for medicinal compositions of the invention are natural products and have a low molecular weight of less than 500, its side effect is by far smaller compared with that of conventional drags.

In case of using the above vitamin K's for the purpose of treatment, formulation into an appropriate form of composition can be prepared. Therefore, in the invention are also provided medicinal compositions, optionally combined formulation with a pharmaceutically acceptable excipient, containing the above vitamin K's and having an effect of treatment for Alzheimer disease and the like.

Therefore, utilizing the medicinal compositions of the invention, treatment for many diseases associated with dementia besides Alzheimer senile dementia can be carried out without being accompanied by a severe side effect.

Meanwhile, although the mechanism of the effect of potentiating nerve growth factor activity of vitamin K's, which are the active ingredients used in the medicinal compositions of the invention, is not necessarily clear, it is presumed that protein kinase A (PKA) is activated, followed by amplification of signal transduction via MAPK (mitogen-activated protein kinase) (see FIG. 4).

### BRIEF DESCRIPTION OF THE DRAWINGS

FIG. 1 is a graphical figure showing the effects of potentiating NGF activity by vitamin K1 and vitamin K2 in the presence of low dose NGF.

FIG. 2 is a graphical figure showing the effects of potentiating NGF activity by the different amounts of vitamin K1 and vitamin K2.

FIG. 3 is a photographic figure showing the appearances of neurite elongation by vitamin K1 and vitamin K2.

FIG. 4 is a graphical figure showing the activities of vitamin K1 and vitamin K2 in the presence of PKA inhibitor, PD98059 or chelerythrine chloride.

### MODE FOR CARRYING OUT THE INVENTION

Vitamin K's used as an active ingredient in the medicinal composition of the invention is not particularly limited as long as they have an effect of potentiating nerve growth factor activity, and illustrative are as represented in the following formulae vitamin K1 (phylloquinone) 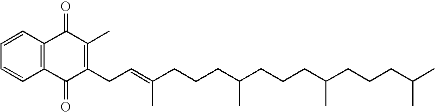
and vitamin K2 (menaquinone; n=1-13) 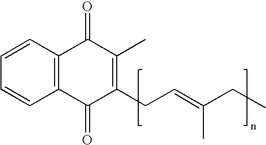
and vitamin K3-K7, as well as hydroquinone derivatives thereof and derivatives such as esters, anhydrides, alkylated compounds, oxidation compounds and reduction compounds thereof.

Medicinal compositions using vitamin K's in the invention can be used as a medicinal composition mixed with one or more carriers which are pharmaceutically acceptable and can be made a known preparation form. Illustrative of pharmaceutically usable carriers are, for example, fillers, extenders, binders, disintegrators, surface active agents, lubricants and moisturizing agents. The representative examples of forms of a pharmaceutical composition include tablets, granules, powders, pills, suppositories, capsules, syrups, emulsions, suspensions, liquids, injections, pastes, ointments, creams, gels, gelatinized creams, lotions, fomentations, plasters, liniments, aerosols, buccals, eye drops, nasal drops, etc., and can stably be administered systemically or locally as well as orally or parenterally.

In case of formulating solid preparations such as tablets and granules, for example, as excipients carbohydrates such as lactose, sugar, glucose, mannitol and sorbitol, starches such as corn starch, potato starch and dextrin, micro-crystalline cellulose, gum arabic, dextrin, pullulan, light anhydrous silisic acids, aluminum silicate, magnesium metasilycate aluminate, magnesium silicate, calcium phosphate, calcium carbonate, calcium sulfate, etc., as disintegrators, carboxymethyl cellulose, sodium carboxymethyl cellulose, calcium carboxymethyl cellulose, hydroxypropyl cellulose, crystalline cellulose, ethyl cellulose, sodium carboxymethyl starch, sodium croscarmellose, etc., as binders, polyvinyl pyrrolidone, polyvinyl alcohol, hydroxypropyl cellulose, etc., as lubricants talc, stearic acid, magnesium stearate, calcium stearate, etc., and others such as polyethylene glycols, propylene glycols and dyes can optionally be mixed. Further, if desired, prepared are solid preparations applied by a usual coating such as a sugar coated solid preparation, a gelatin encapsulated enteric solid preparation, a film coated solid preparation, a double-layer solid preparation or a multilayer solid preparation.

Also, in case of formulating pills, they can be prepared by a known method using a known carrier. As such a carrier, an excipient such as glucose, lactose, starch, cocoa butter, hardened vegetable oil, kaolin, talc or the like, and a disintegrator such as laminaran agar and the like can optionally be mixed.

Also, in case of formulating suppositories, they can be prepared by a known method using a known suppository base. Such a suppository base includes a lipophilic base, an aqueous base, an emulsified base or the like, and they can be used by an appropriate selection. Examples of such a suppository base include cocoa butter, hydrogenated peanut oil, hydrogenated coconut oil, polyethylene glycol, Monolen, Tulen, Prulonic or the like. Further, additives such as an antihistamine, local astringent, sulfa drug, antibiotic, wound healing drug, surface active agent, vitamin, crude drug extract, bile acid, antiseptic, excipient, sorbefacient, amino acid or the like can optionally be mixed.

Also, as a base used for hard capsules or soft capsules in a capsule formulation, a plastisizer such as gelatin, glycerin, sorbitol, propylene glycol, sucrose, gum arabic or the like, a color such as dye, titanium oxide or the like, a preservative such as a methyl, ethyl or propyl p-hydroxybenzoates (parabens) or the like, an aromatic substance and other excipient, etc., are optionally used, whereby they can be formulated.

Also, in case of formulating injections, they can be prepared by a known method using a known dilution agent and the like. As for an injection a septic liquid, emulsion and suspension are used, whereby it is preferable to make them isotonize with the blood. Illustrative of a dilution agent used to make them informs of liquids, emulsions and suspensions are, for example, water, an aqueous lactic acid solution, ethyl alcohol, propylene glycol, polyoxyethyl sorbitan fatty acid ester, etc., and a sufficient amount of sodium chloride, glucose or glycerin to prepare an isotonic solution, a solubilizing agent, a buffer substance, a soothing agent, additionally a color, a preservative, a perfume, a flavoring, a sweetner, etc., are optionally used, whereby it can be formulated.

Also, as formulations of syrups, emulsions, suspensions and liquids, a non-ionic active agents such as water, ethanol, glycerin, sorbitol, polyethylene glycol, propylene glycol, glycerol monostearate, polyoxy stearate, lauromacrogol, sorbitan oleate, polysorbate 80 or sucrose fatty acid ester, an anionic active agents such as stearyl triethanolamine or sodium lauryl sulfate, and a cationic active agents such as benzalkonium chloride or benzethonium chloride as a solubilizing agent or an emulsifying agent, or a surface active agent such as the above non-ionic active agent, anionic agent or cationic agent, a polyvinyl type compound such polyvinyl alcohol or polyvinyl pyrrolidone, a cellulose derivative such as sodium carboxymethyl cellulose, methyl cellulose, hydroxymethyl cellulose, hydroxyethyl cellulose or hydroxypropylmethyl cellulose, additionally gum arabic, gelatin or the like as a dispersing agent, aluminum magnesium silicate, colloidal aluminum magnesium silicate hydrate, bentonite, kaolin or microcrystalline cellulose as a viscous agent, parabens, benzalkonium chloride or benzethonium chloride as a preservative, furctose, invert sugar, cocoa, citric acid, ascorbic acid, fruit juice as a flavoring or a sweetner, and the other excipients, etc., are appropriately used, whereby they can be formulated.

Also, the above liquids can be made to an aerosol composition by mixing a usual propellant agent. As the propellant agent, illustrative are dimethyl ether, a liquefied petroleum gas, nitrogen gas, nitrous oxide gas, carbon dioxide gas, CFCs substitute, etc., which are conventionally used for aerosols. A compressed air can also be used without using a propellant agent. Further, a mixed gas of these can be used. If desired, an aromatic substance or an ingredient conventionally used in a drug for external use, for example, such as 1-menthol, tocopherol acetate, furthermore, oil including a plant oil, an animal oil or the like such as castor oil or squalene are appropriately used, whereby they can be formulated.

Also, in case of formulating ointments, they can be prepared by a known method using a known ointment base. As the ointment base, illustrative are higher fatty acids or esters thereof such as adipic acid, myristic acid, palmitic acid, stearic acid, oleic acid, adipate, palmitate, diethyl sebacate, hexyl laurate and cetyl isooctanoate, waxes such as whale wax, beeswax and Sercin, surface active agents such as polyoxyethylene alkylether phosphate, higher alcohols such as cetanol, stearyl alcohol and cetostearyl alcohol, silicon oils such as dimethylpolysiloxane, methylphenyl polysiloxane, glycolmethyl polysiloxane and silicon-glycol copolymer, hydrocarbons such as propylene carbonate, diisopropyl adipate, crotamiton, Azone and pyrothiodecane, moisturizing agents such as glycerin, propylene glycol, butylene glycol and sorbitol, anti-eruption agents and other additives, and the preparation can be made by an appropriate formulation therefrom.

Also, in case of formulating creams, they can be prepared by a known method using a known cream base. Examples include as the cream base higher fatty acid esters such as myristate, palmitate, diethyl sebacate, hexyl laurate and cetyl isooctanoate, lower alcohols such as ethanol and isopropanol, hydrocarbons such as liquid paraffin and squalene, polyalcohols such as propylene glycol and 1,3-butyleneglycol, higher alcohols such as 2-hexyldecanol, cetanol and 2-octyl decanol, as emulsifying agents polyoxyethylene alkyl ethers, fatty acid esters, polyetylene glycol fatty acid esters, etc., as antiseptics p-hydroxy benzoic acid esters, as sorbefacients propylene carbonate, diethyl cebacate, diisopropyl adipate, crotamiton, Azone and pirothiodecane, anti-eruption agents and other additives, and the preparation can be made by an appropriate formulation therefrom.

Also, in case of formulating gels, they can be prepared by a known method using a known gel base. As the gel base, illustrative are lower alcohols such as ethanol and isopropyl alcohol, gelatinizing agents such as water, carboxyvinyl polymer, hydroxyethyl cellulose, hydroxypropyl cellulose, methyl cellulose, ethyl cellulose, carboxymethyl cellulose and propyleneglycol alginate, neutralizing agents such as triethanolamine, diisopropanolamine and sodium hydroxide, surface active agents such as sorbitan sesquioleate, sorbitan trioleate, sorbitan monooleate, sorbitan monostearate, sorbitan monolaurate, polyethlene glycol monostearate, polyoxyethylene nonylphenyl ether, polyoxyethylene cetyl ether and polyoxyethylene lauryl ether, sorbefacients such as propylene carbonate, diethyl cebacate, diisopropyl adipate, crotamiton, Azone, propylene glycol and pirothiodecane, anti-eruption agents and other additives, and the preparation can be made by an appropriate formulation from these.

Also, in case of formulating gelatinized creams, the above creams were added with a gelatinizing agent such as carboxyvinyl polymer, hydroxyethyl cellulose, hydroxypropyl cellulose, methyl cellulose, ethyl cellulose or carboxymethyl cellulose, adjusting the pH to 4-9, preferably 5-7 by adding a neutralizing agent such as diisopropanolamine, triethanolamine or sodium hydroxide to give the preparation.

Also, in case of formulating fomentations, they can be prepared by a known method using a known fomentation base. Illustrative as thickeners are aqueous synthetic polymers such as sodium polyacrylate, polyacryic acid, Poval, polyvinyl pyrrolidone, polyethylene oxide and polyvinyl methacrylate, natural products such as gum arabic, starch and gelatin, methyl cellulose, hydroxypropyl cellulose, alginic acid, sodium alginate, sodium carboxymethyl cellulose, etc., and further illustrative are wetting agents such as urea, glycerin, propylene glycol, butylene glycol and sorbitol, fillers such as kaolin, zinc oxide, talc, titanium, bentonite, epoxy resins, organic acids (fillers such as tartaric acid, maleic acid, maleic anhydride and succinic acid), calcium, magnesium and aluminum, solubilizinfg agents such as water, propylene carbonate, crotamiton and diisopropyl adipate, tackifying agents such as rosin, ester gum, polybutene and polyacrylate, additionally anti-eruption agents such as diphenhydramine hydrochloride, chloramphenyramine maleate, glycyrrhizic acid, dexamethasone, betamethasone, fluocinolone acetonide, and additives such as salicylic acid, methyl salicylate, glycol salicylate, 1-menthol, camphor, nonylacid vanillil amide, thymol, pepper extract, mint oil, Azone and pirothiodecane, and the preparation can be made by an appropriate formulation therefrom.

Also, in case of formulating plasters, they can be prepared by a known method using a known plaster base. Illustrative as the plaster base are polymer bases such as acrylic type compositions which are copolymers of methacrylate, acrylonitrile, and vinyl monomers such as vinyl acetate or vinyl propionate, silicone resin, polyisobutylene rubber, polyisoprene rubber, natural rubber, acrylic rubber, styrene-butadiene-styrene block copolymer and styrene-isoprene-styrene block copolymer, oils or higher fatty acids such as almond oil, olive oil, camellia oil, bersic oil, olein oil, liquid paraffin and polybutene, adhesive agents such as rosin, rosin-denaturated maleic acid and hydrogenated rosin ester, anti-eruption agents, and additives such as d1-camphor, 1-menthol, thymol, 4-hydroxy-3-methoxybenzyl nonylic acid amide, pepper tincture, mint oil, crotamiton, peppermint oil, Azone and pirothiodecane, and the preparation can be made by an appropriate formulation therefrom.

Also, in case of formulating liniments, they can be prepared by a known method using a known liniment base. Illustrative as the liniment base are alcohols such as ethanol, propanol, iso-propanol, polyethylene glycol, propylene glycol and butylene glycol, fatty acid esters such as each ester of adipic acid, sebacic acid or myristic acid, surface active agents such as polyoxyethylene alkyl ether, neutralizing agents, viscosity donating agents such as methyl cellulose, carboxyvinyl polymer and hydroxypropyl cellulose, anti-eruption agents, and additives such as salicylic acid, methyl salicylate, glycol salicylate, 1-menthol, camphor, mint oil, pepper extract, 4-hydroxy-3-methoxybenzyl nonylic acid amide, thymol, crotamiton, peppermint oil, Azone, propylene carbonate, diisopropyl adipate and pirothiodecane, and the preparation can be made by an appropriate formulation therefrom.

In case of using a medicinal composition of the invention, more particularly, the dose of vitamin K's being different depending on the sex, body weight, age and the specific disease conditions of a patient, usually they can be administered in doses between 0.1 and 1000 mg/kg and in one or more times per day. The content of vitamin K's in the composition is 5-1000 mg. The ratio in the total composition is 0.005-50 wt. %, preferably 0.01-40 wt. %, in particular preferably 0.1-25 wt. %. Taking said constitution, the medicinal compositions of the invention can be used as a therapeutic agent of disease such as Alzheimer disease because they potentiate NGF activity and neurite elongation.

### EXAMPLE

In the following, the invention is explained in more detail by the examples without limiting the invention to these examples.

### Example 1

Experiments showing effect of potentiating nerve growth factor activity of vitamin K's and action mechanism thereof

(1) Experimental Method

a) Bioassay for Neurite Elongation

Cells (PCD12 cells) were harvested and plated on a complete medium of collagen-coating tissue incubation dish in the density of 1.3×10⁴ cells/cm². After 24 hours, NGF (0-50 ng/ml) and 100 μg/ml (the maximum concentration wherein toxicity against PCD12 cells is not observed) of vitamin K1 or vitamin K2 (n=4) were added to the cells. Vitamin K1 and vitamin K2 were dissolved in methanol and the solution was added to the cells at the concentration of less than 1% v/v relative to the medium, which concentration had been demonstrated to leave no effect on PCD12 cells.

After 48 hours of incubation, the ratio of cells having neurites was examined using a phase-contrast microscope under ×200 magnification. The number of neurites which elongate more than 2 times larger compared with a cell body was counted. Each measurement in the data is the mean value calculated from examining random 6 plats in duplicate experiments, whereby at least 100 cells/point was counted for calculation.

b) Effect of Each Inhibitor

In order to study the action mechanism of vitamin K's, influence of MAPK kinase inhibitor (PD98059), protein kinase A (PKA) inhibitor, and protein kinase C (PKC) inhibitor (chelerythrine chloride) on the effects for potentiating neurite elongation of vitamin K's in PCD12 cells were studied.

Pre-incubation of cells was carried out for 1 hour in the presence or in the absence of the inhibitor before treatment of NGF or vitamin K compound. After 48 hours of incubation, the ratio of cells having neurites was examined.

(2) Results

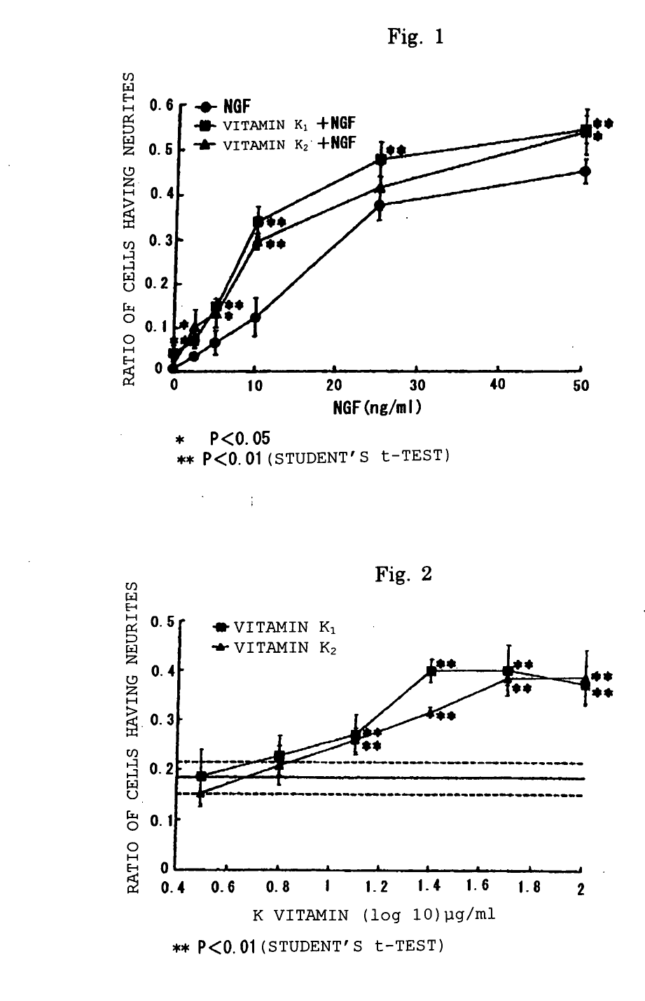

As shown in FIG. 1, by the addition of 100 μg/ml of vitamin K1 or vitamin K2 in the presence of low dose of NGF (0-50 ng/ml), the effect of potentiating neurite elongation in PCD12 cells was confirmed. Namely, by the addition of vitamin K's (100 μg/ml), vitamin K1 and vitamin K2 showed respectively about 1.8 times and about 2.4 times of the effect of potentiating neurite elongation compared with the negative control group added with NGF 10 ng/ml. As the results, the concentration-response curve between the concentration of NGF and the ratio of cells having neurites shifted toward left side.

Also, the effect of potentiating neurite elongation for PCD12 cells with NGF (10 ng/ml) was potentiated by vitamin K's in a concentration dependent manner (FIG. 2).

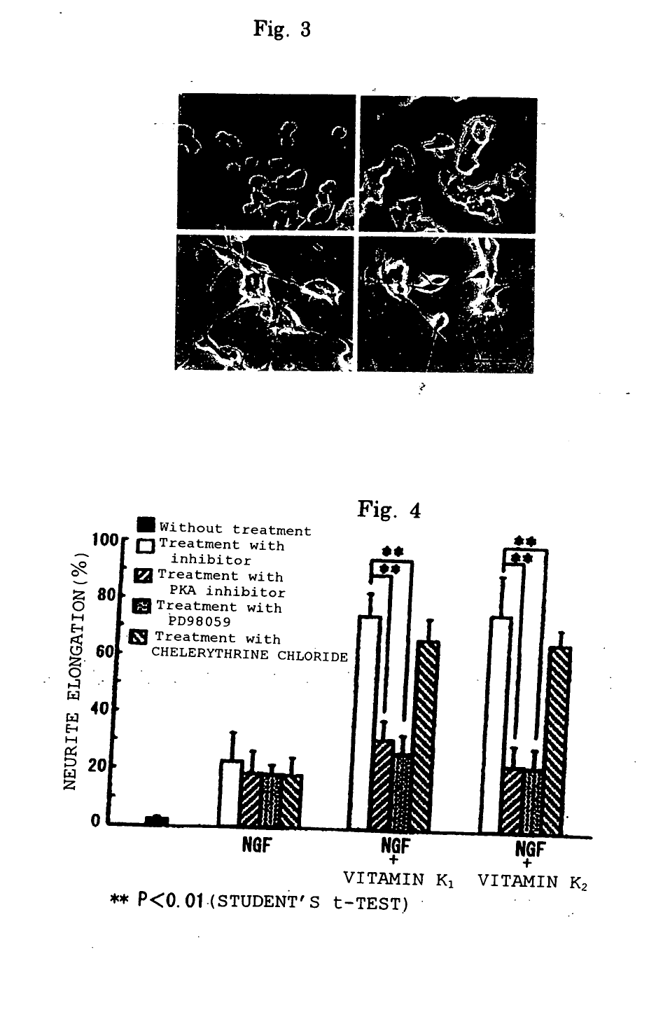

The morphological change of PCD12 cells by each of treatment is shown in FIG. 3. Treatment with vitamin K1 or vitamin K2 (100μg/ml) together with NGF (10 ng/ml) remarkably increased the number of cells having neurites.

Based on the above results, it became clear that vitamin K's have effect of potentiating nerve growth factor activity.

The results of the experiments studying the action mechanism of vitamin K's are shown in FIG. 4.

The neurite elongation in PCD12 cells potentiated by vitamin K's was significantly inhibited by 5 nM of PKA. Further, the potentiation of vitamin K's was almost completely inhibited by pretreatment of cells with 10 μM of PD98059, MAPK kinase specific inhibitor. Contrasting to this, a significant inhibition to neurite elongation in cells treated with PKC inhibitor, chelerythrine chloride, was not observed.

Based on the above results, it was presumed that the effect of potentiating nerve growth factor activity of vitamin K's is due to activation of protein kinase A (PKA), followed by amplification of signal transduction via MAPK (mitogen-activated protein kinase).

In the following, the formulation examples of preparations prepared by using vitamin K's of the invention, more particularly, vitamin K1 or vitamin K2 (n=4), are described. Meanwhile, these preparation formulation examples are only for illustration, and it is to be understood that various preparation formulations can be made according to a known method.

### Example 2

Preparation Formulation 1

An aqueous injection is prepared by the following mix formulation.

| | |
|---|---|
| Vitamin K1 or Vitamin K2 | 1.0 g |
| Benzyl alcohol | 2.0 g |
| Nicotinamide | 3.0 g |
| Propylene glycol | 40.0 g |
| Distilled water | 100 ml |

### Example 3

Preparation Formulation 2

An injection of lipid emulsion is prepared by the following mix formulation.

| | |
|---|---|
| Vitamin K1 or Vitamin K2 | 1.0 g |
| Soybean oil (Japanese Phrmacopeia) | 21.0 g |
| Purified soybean phospholipid | 2.5 g |
| Glycerin | 5.0 g |
| Distilled water | 175 ml |

### Example 4

Preparation Formulation 3

A tablet is prepared by the following mix formulation (total amount: 100 wt. %).

| | |
|---|---|
| Vitamin K1 or Vitamin K2 | 25.0 wt. % |
| Fumaric acid | 10.0 wt. % |
| Calcium hydrogenphosphate | 45.0 wt. % |
| Lactose | 24.0 wt. % |
| Talc | 1.0 wt. % |

The tablet was applied by spray coating of a coating liquid consisting of ethyl cellulose, polyvinyl pyrrolidone K30, talc and ethyl alcohol according to a conventional method to prepare a sustained release tablet.

### Example 5

Preparation Formulation 4

A suppository is prepared by the following mix formulation (the total amount, 100 wt. %).

| | |
|---|---|
| Vitamin K1 or Vitamin K2 | 5.0 wt. % |
| Propylene glycol | 6.0 wt. % |
| White beeswax | 10.0 wt. % |
| Sorbitan sesquioleate | 4.49 wt. % |
| Medium chain fatty acid triglyceride | 74.5 wt. % |
| Dibutyl hydroxy toluene | 0.01 wt. % |

### Example 6

Preparation Formulation 5

An ointment is prepared by the following mix formulation (the total amount, 100 wt. %).

| | |
|---|---|
| Vitamin K1 or Vitamin K2 | 5.0 wt. % |
| Propylene glycol | 6.5 wt. % |
| Isopropyl myristate | 5.5 wt. % |
| White petrolatum | 83.0 wt. % |

### Example 7

Preparation Formulation 5

A liniment is prepared by the following mix formulation (total amount: 100 wt. %).

| | |
|---|---|
| Vitamin K1 or Vitamin K2 | 3.0 wt. % |
| Ethanol | 38.0 wt. % |
| 2-hydroxy-4-methoxybenzophenone | 0.5 wt. % |
| Propylene glycol | 13.0 wt. % |
| Methyl cellulose | 0.8 wt. % |
| Ethyl sebacate | 3.0 wt. % |
| Purified water | appropriate |
| Sodium hydroxide | 0.07 wt. % |

### Example 8

Preparation Formulation 7

A gel is prepared by the following mix formulation (the total amount, 100 wt. %).

| | |
|---|---|
| Vitamin K1 or Vitamin K2 | 3.0 wt. % |
| Diisopropyl adipate | 3.0 wt. % |
| Ethanol | 38.5 wt. % |
| Carboxyvinyl polymer | 2.0 wt. % |
| Purified water | appropriate |
| Hydroxypropyl cellulose | 2.0 wt. % |
| Propylene glycol | 17.0 wt. % |
| Diisopropanolamine | 2.5 wt. % |

### Example 9

Preparation Formulation 8

A gelatinized cream is prepared by the following mix formulation (the total amount, 100 wt. %).

| | |
|---|---|
| Vitamin K1 or Vitamin K2 | 3.0 wt. % |
| Isopropyl myristate | 11.0 wt. % |
| Ethanol | 6.0 wt. % |
| Carboxyvinyl polymer | 1.5 wt. % |
| Purified water | appropriate |
| Polyoxyethylene (55) monostearate | 1.0 wt. % |
| Coconut oil fatty acid diethanolamide | 4.0 wt. % |

### Example 10

Preparation Formulation 9

A cream is prepared by the following mix formulation (the total amount, 100 wt. %).

| | |
|---|---|
| Vitamin K1 or Vitamin K2 | 5.0 wt. % |
| Cetyl alcohol | 12.0 wt. % |
| Stearyl alcohol | 2.5 wt. % |
| Glyceryl monostearate | 6.0 wt. % |
| 1,3-butylene glycol | 13.0 wt. % |
| Purified water | appropriate |

### Example 11

Preparation Formulation 10

A fomentation is prepared by the following mix formulation (total amount: 100 wt. %).

| | |
|---|---|
| Vitamin K1 or Vitamin K2 | 3.0 wt. % |
| Gelatin | 6.0 wt. % |
| Aluminum silicate | 11.0 wt. % |
| Polyvinyl alcohol | 4.5 wt. % |
| Purified water | appropriate |
| Glycerin | 28.0 wt. % |
| Carboxymethyl cellulose | 3.0 wt. % |

### Example 12

Preparation Formulation 11

A plaster is prepared by the following mix formulation (total amount: 100 wt. %).

| | |
|---|---|
| Vitamin K1 or Vitamin K2 | 3.0 wt. % |
| Styrene-isoprene-styrene block copolymer | 24.5 wt. % |
| (Kalyflex TR1107, manufactured by Shell | |
| Kagaku K. K.) | |
| Liquid paraffin | 43.5 wt. % |
| Hydrogenated rosin ester | 29.0 wt. % |

### Example 13

Preparation Formulation 12

A nasal drops are prepared by the following mix formulation.

| | |
|---|---|
| Vitamin K1 or Vitamin K2 | 10 mg |
| Propylene glycol | 20 mg |

Distilled water: appropriate (Total amount is made 100 ml.)

### INDUSTRIAL APPLICABILITY

According to the invention, utilizing the medicinal compositions containing vitamin K's as an active ingredient, treatment for many disease associated with dementia except Alzheimer senile dementia can be carried out without accompanying a severe side effect. Therefore, the invention greatly contributes to pharmaceutical industries and related industries.

1. A medicinal composition containing an effective amount of vitamin K and/or its derivative showing an effect of potentiating nerve growth factor activity.

2. The medicinal composition according to claim 1, wherein vitamin K and/or its derivative is one or more selected from vitamin K1, vitamin K2 or derivatives thereof.

3. The medicinal composition according to claim 1, wherein the amount of vitamin K and/or its derivative showing an effect of potentiating nerve growth factor activity is 0.005-50 wt. %.

4. A method for treatment of disease associated with dementia comprising administering to a patient an effective amount of the medicinal composition according to claim 1.

5. The method of claim 4, wherein the disease associated with dementia is selected from the group consisting of Alzheimer senile dementia, juvenile Alzheimer dementia, Pick disease, Huntington chorea, Parkinson disease, spinocerebellar degeneration, progressive supranuclear palsy, intractable epilepsy, multiple cerebral infarction, Binswanger type subcortical encephalopathy, chronic subdural hematoma, multiple sclerosis, collagen disease, primary brain tumor, metastatic brain tumor, limbic encephalitis, manic-depressive psychosis, schizoid and reactive psychosis.
